(19) United States
(12) Patent Application Publication
(10) Pub. No.: US 2005/0070028 A1
(43) Pub. Date: Mar. 31, 2005

(54) Method for manufacturing antibody detector and method for detecting antibody

(75) Inventors: Seiichiro Ito, Tokyo (JP); Junko Tanoue, Tokyo (JP); Fumiko Yasukawa, Tokyo (JP)

Correspondence Address: Reed Smith LLP, 3110 Fairview Park Drive, Suite 1400, Falls Church, VA, 22042-4503, US

(73) Assignee: Hitachi Software Engineering Co., Ltd.

(21) Appl. No.: 10/935,162

(22) Filed: Sep. 8, 2004

(30) Foreign Application Priority Data
Sep. 25, 2003 (JP) 2003-333841

(51) Int. Cl. G01N 33/543

(52) U.S. Cl. 436/518

(57) ABSTRACT

In order to specifically and high-sensitively measure multiple antibodies, an antibody detector utilizes the specificity of an antigen-antibody reaction. The antibody detector comprises a plurality of polypeptide units, each of which is partly or entirely epitope, that are connected, with or without a spacer, and that are immobilized on a carrier.

### BACKGROUND OF THE INVENTION

1. Field of the Invention

The present invention relates to an antibody detector used to specifically detect and quantify only a single type of antibody in a solution in which multiple antibodies are mixed, and to a method for manufacturing such antibody detector. Further, the present invention relates to a method for detecting antibodies using such antibody detector.

2. Background Art

Antibodies are those proteins produced inside a living body through an immune reaction as a result of stimulation of antigens, and which have an activity to specifically bind with the antigens. An antibody is capable of specifically reacting with an antigen to achieve aggregation and sedimentation, or neutralization of the toxicity of the antigen, providing the living body with immunity against the antigen. The antibodies have the property of specifically and strongly binding with antigens, so that the antibodies are widely used in highly sensitive detection of various antigens.

Although relatively low-molecular polysaccharides, nucleic acids, and lipids have the possibility of becoming antigens, proteins or other substance with heavier molecular weights are more likely to become antigens. The portion of the antigen to which an antibody binds is referred to as an epitope. Generally, a single type of protein has epitopes at multiple locations. The antibodies that recognize the same protein have different binding capacities and recognition abilities if their epitopes are different. Some antibodies lose or maintain their binding capacities as a result of posttranslational modification such as phosphorylation, glycosylation, and oxidation-reduction. Accordingly, it is expected that antibodies that recognize the same proteins have different functions in the immune system of a living body. There is a growing demand for measurement of specific antibody types, in addition to quantification of antibody groups that recognize proteins.

Mass purification of a single type of protein is technically difficult. In experiments for detecting antibodies, the frequency of antibodies that recognize different antigens nonspecifically interacting with each other is high. Generally, proteins of a single type have epitopes at multiple locations, and it is not suitable to use a full-length protein as an antigen for measuring a single type of antibodies. Therefore, in principle, it is desirable to use polypeptides of 10 or so amino acids as antigens, which are considered epitopes. Technologically, polypeptides of 10 or so amino acids can be artificially synthesized in large quantities, and the number of antibodies that interact nonspecifically can be minimized using the polypeptides.

To date, known antibody detecting methods include one by which antigens such as proteins or polypeptides are bound using microplates and the like as a carrier, and detection is conducted using the EIA method or the like.

Recently, techniques are being developed that enable simultaneous measurement of multiple items using flat chips or membranes as carriers on which biopolymers such as nucleic acids, antibodies, or antigens are immobilized. In particular, the SAT (Suspension Array Technology) to which the principle of a flow cytometer is applied is gaining attention in terms of ease of use, cost, the short length of time required, and the high reliability of detection results. JP Patent Publication (Kokai) No. 2002-311027 A discloses beads that can emit multiple fluorescent lights and that can be recognized by a flow cytometer, and the flow cytometer used for that purpose. The SAT is a technique that employs multiple cellular-sized microbeads whose emission of light is varied by using different blending ratios of multiple dyes. After the multiple beads are reacted in the same solution sample, the quantity of fluorescence on the surface of each microbead is measured to enable simultaneous measurement of multiple items. The microbeads are coated with a carboxyl or amide group, so that they can immobilize biopolymers such as nucleic acids, antibodies, or antigens.

FIG. 6 schematically shows an antibody detector that uses a conventional microbead as a carrier. A polypeptide amino acid is connected to a carboxyl group on a polystylene microbead via a spacer. The spacer is used here so that, when a protein specifically reacts with the polypeptide, the protein, which is a large molecule, can be prevented from being affected by steric hindrance.

FIG. 7 shows the overview of a fluorescent microbead array system. Of the two microbeads on which two different types of peptides are fixed, only microbead 1 specifically reacts with an antibody as a measurement object. The antibody is then reacted with a biotinylated antibody labeled with a fluorescent material such as avidin-PE, and then fluorescent measurement is conducted using Luminex (trade name). In contrast, microbead 2 that did not specifically react with the antibody does not emit fluorescent light.

JP Patent Publication (Kokai) No. 5-91879 A (1993) can be cited as disclosing prior art relating to the present invention.

### SUMMARY OF THE INVENTION

In general, since proteins have multiple epitopes, it is not suitable to use full-length proteins as antigens for measuring a single type of antibodies. However, polypeptides of 10 or so amino acids, which are considered epitopes, when used as antigens, are unstable in terms of physical properties, as compared with a full-length protein. Also, the detection sensitivity of such polypeptides is generally low. When conducting an antigen-antibody reaction in detection experiments, it is expected that if the physical distance between the carrier and the antigen fixed on the carrier is short, antibodies cannot come close to the antigen, leading to a lower frequency of contact and failure to form a three-dimensional structure for the antibodies to recognize. This is thought to be a reason for low detection sensitivity. To solve these problems, a method is employed by which a material referred to as a spacer is inserted between the carrier and the antigen to maintain a physical distance before the carrier and the antigen are bound. This, however, more than often than not proves ineffective, as the number of steps of the experiment increases and more time and labor is required.

Presently, in a typical method for detecting antibodies, a microplate and the like are used as a carrier for bonding antigens, and then antibodies are detected using the EIA method, for example. However, detecting multiple antibodies requires a sufficient amount of sample commensurate with the number of antibodies and also time and labor. If the amount of sample is small, the possibility of having to limit the number of antibodies that can be detected increases.

The inventors arrived at the present invention after discovering that the aforementioned problems can be solved by using an antibody detector having a specific structure.

A method for detecting antibodies according to the present invention involves the detection of an antigen using the specificity of an antigen-antibody reaction. The method comprises connecting polypeptide units including epitopes to a carrier, specifically and with high-sensitivity detecting, by applying the SAT technology, multiple antibodies simultaneously in a small amount of sample in which multiple antibodies, such as blood, blood serum, and supernatant of cell culture solution, are mixed, and quantifying the detected antibodies.

In a first aspect, the present invention provides an antibody detector utilizing the specificity of an antigen-antibody reaction. The antibody detector comprises a plurality of polypeptide units, each of which is partly or entirely epitope, that are connected, with or without spacers, and immobilized on a carrier. The carrier may be a flat biochip or microbead. Preferably, the polypeptide unit to be connected on the carrier should consist of 6 to 15 amino acids, and the epitope in the polypeptide unit should preferably consists of 4 to 8 amino acids.

In the present invention, since multiple polypeptide units are connected, the distance between the carrier and the eiptope is maintained sufficiently, so that conventional spacers for excluding steric hindrance during an antigen-antibody reaction are not necessary. However, the spacers may be included. In that case, almost all of the polypeptide units that include epitopes would be involved in an antigen-antibody reaction, which is preferable.

FIG. 1A schematically shows a polypeptide unit used for the present invention. Several to a dozen or so amino acids form a peptide bond to constitute the peptide unit. The peptide unit includes an epitope portion that consists of a specific sequence of several to ten or so amino acids and that specifically reacts with a protein. The peptide unit includes an N terminal of an amino group and a C terminal of a carboxyl group.

FIG. 1B shows a plurality of polypeptide units including epitopes shown in FIG. 1A that are connected and immobilized on a microbead used as a carrier. In some cases, some of the polypeptide units may branch from the main chain. As shown in FIG. 1B, a number of the epitope portions that specifically react with a protein are disposed, while maintaining a certain distance from the microbead, thereby constituting the antibody detector of the present invention.

FIG. 2 shows antibodies having specifically reacted with epitope portions of the antibody detector shown in FIG. 1B. It will be seen that the antibodies efficiently react with the many epitope portions. By thus connecting the polypeptide units including the multiple epitopes to the carrier, (1) the absolute number of portions (epitope portion) that the antibodies recognize increases, and, at the same time, (2) a sufficient distance can be maintained between the carrier and the epitopes becomes sufficient, and steric hindrance that prevents an antigen-antibody reaction can be reduced, thereby enabling high-sensitive detection and measurement of an antigen-antibody reaction.

If the polypeptide units to be connected to the carrier (1) tend to be acidic, (2) tend to be alkaline, (3) are hydrophobic, or (4) form a three-dimensional structure that weakens the antibody recognition, these problems should preferably be solved by adding an amino acid sequence of 1 to 5 amino acids that is not included in the analyte protein.

In a second aspect, the present invention provides a method for manufacturing an antibody detector using the specificity of an antigen-antibody reaction. The method comprises connecting a plurality of polypeptide units, each of which is partly or entirely epitope on a carrier with or without a spacer.

Preferably, when connecting the polypeptide units to the carrier that is coated with a carboxyl or amide groups, EDC (1-Ethyl-3-(3-dimethylaminopropyl) Carbodiimide hydrochloride) should be used as a catalyst to cause a binding reaction. FIG. 3 shows a reaction in which peptide units are bound using EDC. The peptide bonding is performed by causing the amino group terminal of the peptide unit to catalytically react with the carboxyl group on the microbead. The peptide bonding is performed in a chain-reactive manner by causing the amino group terminal of the next peptide unit to catalytically react with carboxyl group terminal of the earlier peptide unit.

Preferably, a flat biochip or spherical bead should be used as the carrier.

Although the number of amino acids in the polypeptide unit connected to the carrier is not limited, preferably there should be 6 to 15 amino acids. Also, the number of amino acids in the epitope in the polypeptide unit is not limited, however, 4 to 8 amino acids are preferred.

In a third aspect, the present invention provides an antibody detection method for detecting an antibody using the specificity of an antigen-antibody reaction. By using the antibody detector of the first aspect of the invention, a single type of antibodies can be specifically and high-sensitively detected and quantified from a solution in which one or more kinds of antibodies are mixed. Especially, the present invention can be effectively applied to a fluorescent microbead array system. The fluorescent microbead array system involves a method for detecting antibodies as shown in FIG. 7, and is being implemented with the trade name of Luminex.

In a more concrete example of the antibody detection method, hybridization reactions can be simultaneously performed in the same solution in which multiple antibodies are mixed. By connecting the aforementioned polypeptide units different from one another to multiple kinds of carriers identifiable upon detection, multiple antibodies can be specifically and high-sensitively detected simultaneously and quantified, even in a small amount of sample.

The present invention enables the simultaneous detection of multiple antibodies in a small amount of sample to be quantified with a higher sensitivity, which has been difficult by conventional methods. By efficiently connecting peptides using EDC, the absolute number of epitopes that antibodies can recognize is simply increased. Moreover, the peptides that are directly bound to the carrier function as a spacer, so that the inhibition of an antigen-antibody reaction can be prevented. By using a carrier to which the SAT technology is applied, multiple antibodies can be specifically detected from the same sample.

The aforementioned peptides are artificially synthesized for use. In light of the efficiency with which the peptides are connected to the carrier, and with which the antibodies recognize the peptides, the higher the degree of refining, the better.

By connecting polypeptide units including multiple epitopes to the carrier, the absolute number of portions that antibodies recognize increases, and, at the same time, a sufficient distance can be maintained between the carrier and the epitopes, and steric hindrance that prevents an antigen-antibody reaction can be reduced, thereby enabling high-sensitive measurement. This allows a higher-sensitivity quantification of the simultaneous detection of multiple antibodies in a small amount sample, which has been difficult by conventional methods. Especially, by applying the present invention to the fluorescent microbead array system, antibodies can be detected with high sensitivity.

### BRIEF DESCRIPTION OF THE DRAWINGS

FIG. 1A schematically shows a peptide unit used in the present invention.

FIG. 1B shows multiple polypeptide units including the epitope shown in FIG. 1A that are connected and immobilized on a microbead used as a carrier.

FIG. 2 schematically shows antibodies having specifically reacted with the epitope portion of the antibody detector shown in FIG. 1B.

FIG. 3 schematically shows a reaction in which peptides are connected to the carrier using EDC.

FIG. 4 shows a table of embodiment results and an illustration of the principle of a fluorescence detection method.

FIG. 5 shows a bar graph of embodiment results.

FIG. 6 schematically shows a method in which a peptide is connected to a carrier using a spacer.

FIG. 7 shows an illustration of the principle of a fluorescence detection method using a fluorescent microbead system.

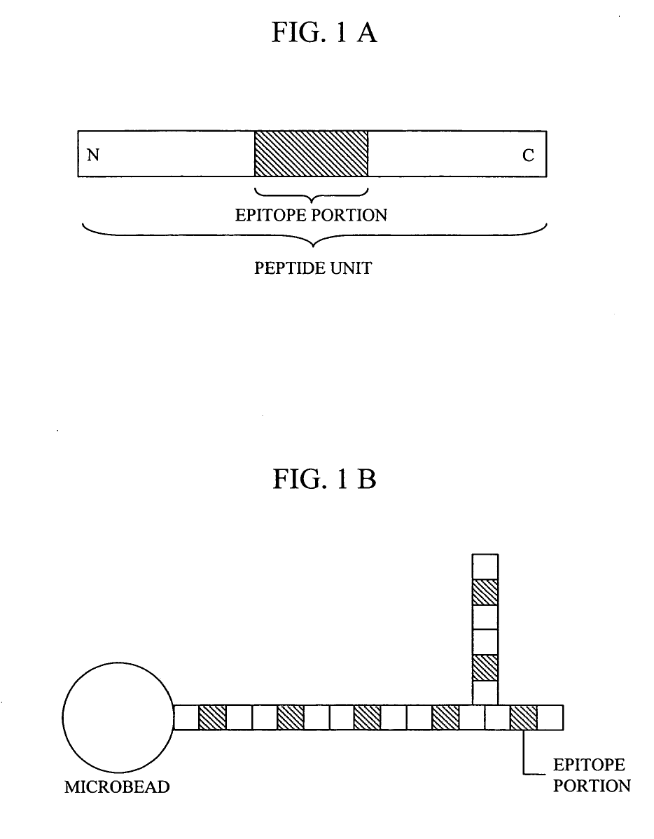

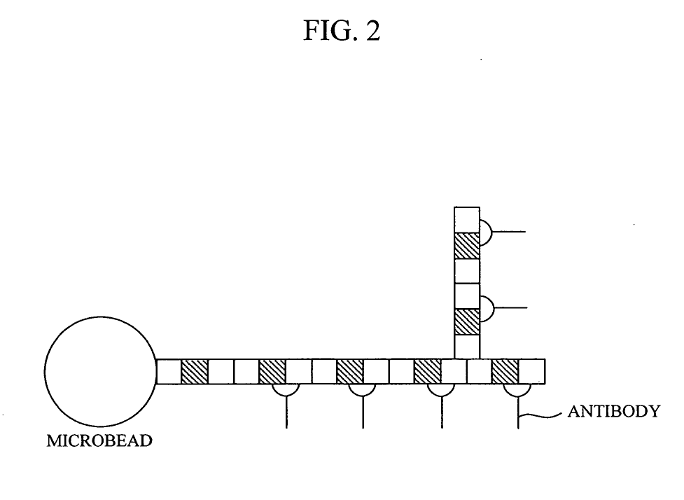

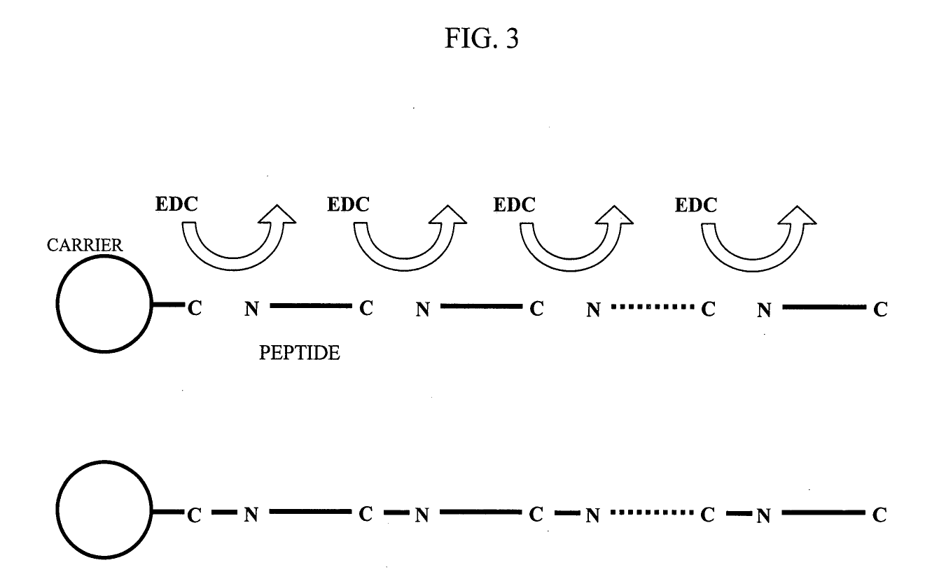

### DESCRIPTION OF THE PREFERRED EMBODIMENTS

While an embodiment of the present invention is described in the following, the present invention is not limited to the embodiment.

(Object)

The effects of simultaneous and high-sensitivity quantification of multiple antibodies in a small amount of sample in accordance with the method of the present invention, which has been difficult by the conventional methods, are proven.

(Measurement System)

In the present embodiment, a fluorescent microbead array system as typified by Luminex (trade name), to which the SAT technology is applied, is used for measurement. In the Luminex system, special beads are used as carriers, so that simultaneous measurement of multiple items is possible. The special beads are made of polystyrene, about 5.6 μm in diameter, and are dyed using multiple fluorescent materials. By changing the content of each of the fluorescent materials, the beads can be identified by the difference of color if they are mixed in the same solution. By having different biopolymers such as antibodies or nucleic acids bind to the beads, the presence or absence of biopolymers that interact with these biopolymers can be measured. Namely, the system can conduct a high-sensitivity, multiple-item measurement of in a small amount of sample.

(Materials)

(1) A peptide A of a specific sequence of 10 amino acids, an antibody A whose antigen is the peptide A, a peptide B of a specific sequence of 11 amino acids which is different from that of the peptide A, and an antibody B whose antigen is the peptide B, are used. The peptide B used here is a polypeptide of 11 amino acids that has the structure of Arg-Pro-Lys-Pro-Gln-Gln-Phe-Phe-Gly-Leu-Met-NH₂. The peptide is generally known as “substance P”.

(2) As a reaction accelerating agent, EDC (1-Ethyl-3-(3-dimethylaminopropyl) Carbodiimide hydrochloride) is used.

(3) As a buffer solution, MES (2-(N-morpholino) ethane sulfonic acid) is used.

(4) As a carrier to which a peptide is bound, a Luminex carboxyl coated bead is used.

(Method)

(1) Preparation of Peptide

The peptides are artificially synthesized for use. In light of the efficiency with which the peptides are connected to the carrier, or the efficiency with which the antibodies recognize the peptides, the higher the degree of refining, the better. The synthesized peptides are dissolved in 0.1 M MES (pH 4.5) so that the final concentration is 0.2 mg/ml. If the peptides are difficult to be dissolved because they are hydrophobic, for example, the peptides may be once dissolved in DMSO and the like, and then dissolved in 0.1 M MES (pH 4.5). Further, if the peptides are expected to be hydrophobic, or extremely acidic or alkaline, based on the amino acid sequence of the peptides, such problems should preferably be solved by adding an amino acid sequence of 1 to 5 amino acids that is not included in the original protein. In the present embodiment, peptides A and B were dissolved in 0.1M MES (pH 4.5) so that the final concentration of the peptides A and B was 0.2 mg/ml.

(2) Covalent Bonding of the Carboxyl Group of the Carrier to the Amide Group of the Peptide

2.5×10⁻⁶ carboxyl-coated beads were suspended in a 50 μl of peptides of 0.2 mg/ml, and then 2.5 μl of EDC of 10 mg/ml was added. The mixture was allowed to stand at room temperature for 30 minutes. Then, the operation of adding a 2.5 μl of EDC of 10 mg/ml and then allowing to stand at room temperature for 30 minutes was performed twice. After washing the carboxyl-coated beads with PBS (pH7.4)-Tween20 (0.05%) twice, the carboxyl-coated beads were re-suspended in PBS (pH7.4)-BSA (10 mg/ml)-Sodium Azide (0.05%) and stored overnight at 4° C. without light. In the present embodiment, the peptides A and B were bound to different carboxyl-coated beads, beads without peptides were also prepared as a negative control by performing the same operation.

(Measurement Using the Luminex Measuring Apparatus)

The peptide-bound beads were put in a sample with a known or unknown antibody concentration and allowed to stand. If antibodies exist in the sample, they should initiate an antigen-antibody reaction with the peptides connected to the beads. The antibodies on the surface of the beads are labeled with a fluorescent material, and the quantity of fluorescence is measured by the Luminex measurement apparatus. In the present invention, three types of beads, namely, beads with peptide A, beads with peptide B and beads without peptides were put in a diluted sample of the antibody B. After fluorescent-labeling with a biotinylated anti-human antibody and avidin-phycoerythrin, the amount of fluorescence was measured by the Luminex measurement apparatus.

(Results)

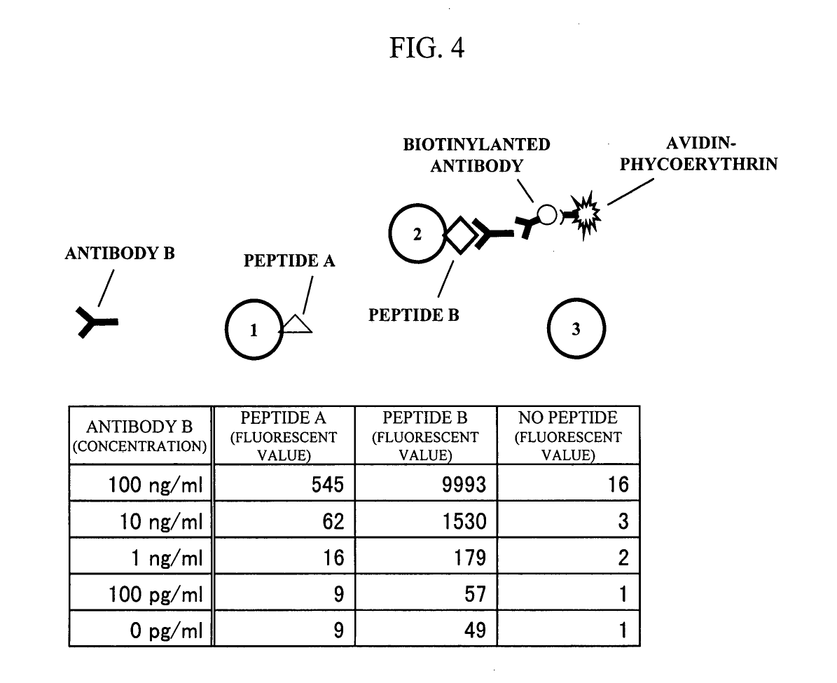

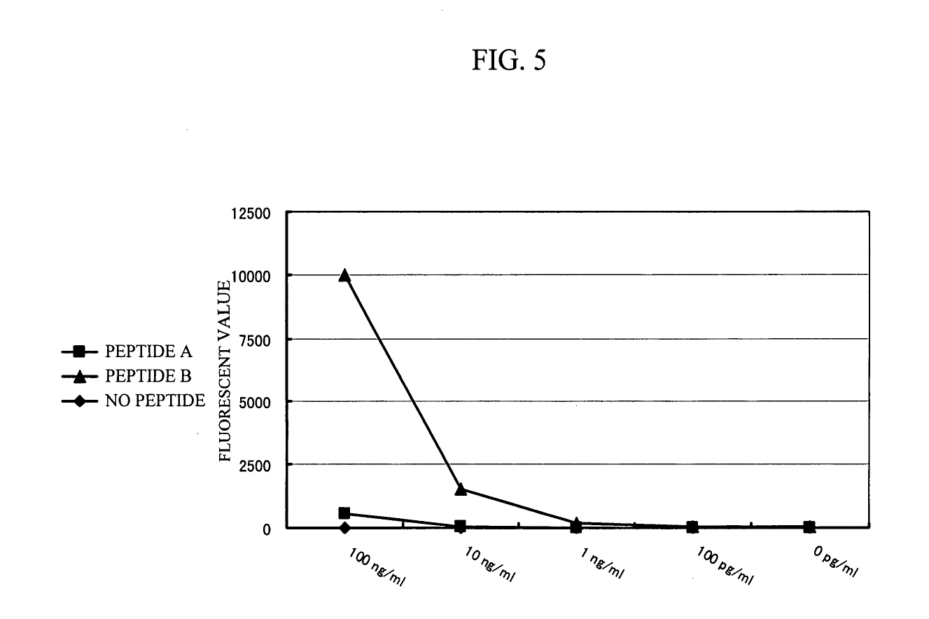

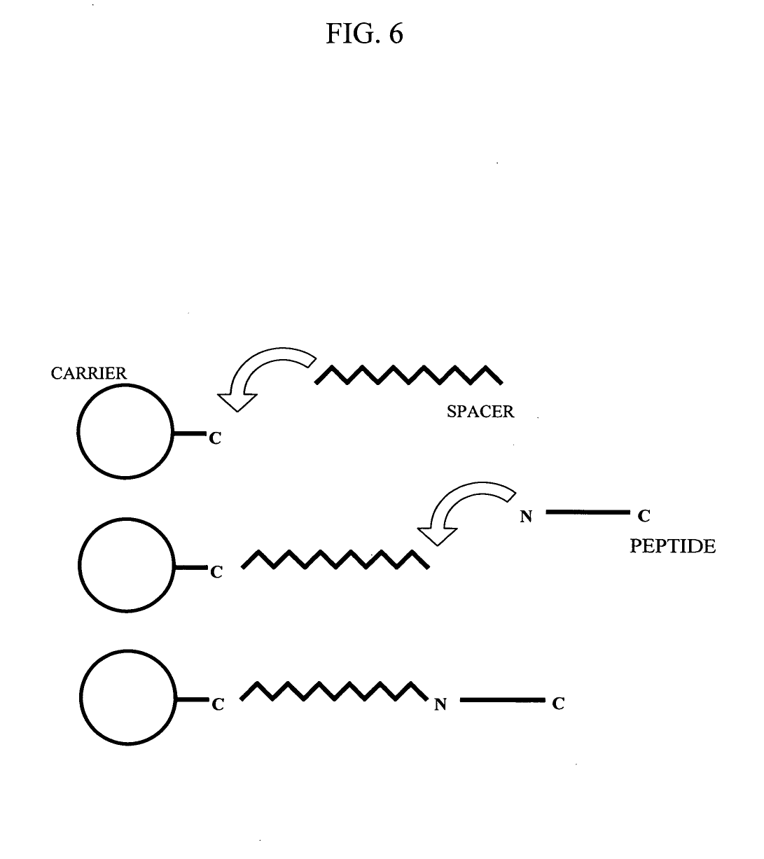

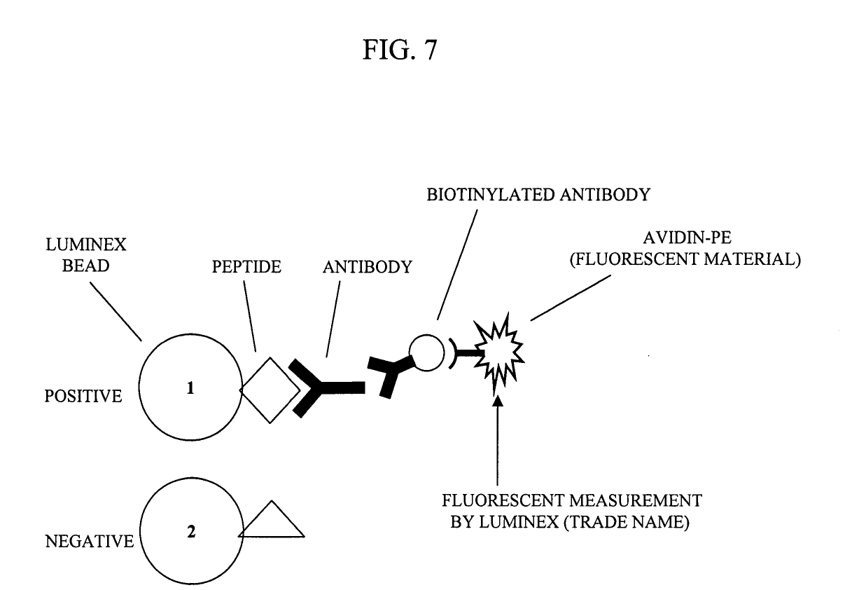

FIG. 4 shows a table of results obtained by the present embodiment. FIG. 5 shows a bar graph of the results shown in FIG. 4. Only the fluorescent values of the beads to which the peptide B was bonded show a fluctuation depending on the concentration of the antibody B. Especially, it will be seen that even for antibodies of lower concentration, the beads of peptide B exhibits certain fluorescence, and that for antibodies of higher concentration, the beads of peptide B show a remarkable fluorescent reaction as compared with the beads of peptide A or the beads without peptides.

When conventional methods for binding proteins to the carrier or binding methods whereby a conventional spacer is placed between the carrier and peptide were tested using the peptides A and B, similar results were not obtained in terms of detection sensitivity and specificity.

Moreover, in a fluorescent measurement with the Luminex system, by optimizing the buffer and the like, a detection sensitivity of about 10 pg/ml in antibody concentration was obtained.

The present invention enables the simultaneous detection of multiple antibodies in a small amount of sample to be quantified with a higher sensitivity, which has been difficult by conventional methods. Especially, by applying the present invention to a fluorescent microbead array system, antibodies can be detected with high sensitivity, thereby making the present invention useful in the fields of medicine and pharmaceutical development.

1. An antibody detector utilizing the specificity of an antigen-antibody reaction, comprising a plurality of polypeptide units, each of which is partly or entirely epitope, that are connected, with or without a spacer, and immobilized on a carrier.

2. The antibody detector according to claim 1, wherein said carrier is a flat biochip or a microbead.

3. The antibody detector according to claim 1, wherein said polypeptide unit to be connected to said carrier comprises 6 to 15 amino acids.

4. The antibody detector according to claim 1, wherein said epitope in said polypeptide unit comprises 4 to 8 amino acids.

5. The antibody detector according to claim 1, wherein if said polypeptide units to be connected to the carrier (1) tend to be acidic, (2) tend to be alkaline, (3) are hydrophobic, or (4) form a three-dimensional structure that weakens the antibody recognition, these problems (1) to (4) are solved by adding an amino acid sequence of 1 to 5 amino acids that is not included in an analyte protein.

6. A method for manufacturing an antibody detector utilizing the specificity of an antigen-antibody reaction, said method comprising connecting a plurality of polypeptide units, each of which is partly or entirely epitope, with or without a spacer, and immobilizing them on a carrier.

7. A method for manufacturing an antibody detector according to claim 6, wherein when connecting said polypeptide units to a carrier coated with a carboxyl or amide group, EDC (1-Ethyl-3-(3-dimethylaminopropyl) Carbodiimide hydrochloride) is used to cause a bonding reaction.

8. The method for manufacturing an antibody detector according to claim 6, wherein said carrier is a flat biochip or microbead.

9. The method for manufacturing an antibody detector according to claim 6, wherein said polypeptide unit to be connected to said carrier comprises 6 to 15 amino acids.

10. The method for manufacturing an antibody detector according to claim 6, wherein said epitope in said polypeptide unit comprises 4 to 8 amino acids.

11. A method for detecting an antibody utilizing the specificity of an antigen-antibody reaction, wherein by using said antibody detector utilizing the specificity of an antigen-antibody reaction, comprising a plurality of polypeptide units, each of which is partly or entirely epitope, that are connected, with or without a spacer, and immobilized on a carrier, only a single type of antibody is specifically and high-sensitively detected from a solution in which one or more types of antibodies are mixed, and is then quantified.

12. The method for detecting an antibody according to claim 11, said method being applied to a fluorescent microbead array system.

13. The method for detecting an antibody according to claim 11, wherein hybridization reactions can be conducted simultaneously in the same solution in which multiple antibodies are mixed, and wherein, by connecting said polypeptide units different from one another to multiple-kinds of carriers that are identifiable upon detection, multiple antibodies can be specifically and high-sensitively detected even in a small amount of samples and then quantified.
